(19) United States
(12) Patent Application Publication
(10) Pub. No.: US 2019/0046118 A1
(43) Pub. Date: Feb. 14, 2019

(54) SENSING GUIDEWIRE WITH TRANSITION TUBE FOR ELECTRICAL INTERCONNECTION

(71) Applicant: KONINKLIJKE PHILIPS N.V., Eindhoven (NL)

(72) Inventors: David Holt BURKETT, Panama City Beach, FL (US); Waheed ABDUL, San Diego, CA (US)

(21) Appl. No.: 16/059,853

(22) Filed: Aug. 9, 2018

Related U.S. Application Data
(60) Provisional application No. 62/543,818, filed on Aug. 10, 2017.

(51) Int. Cl. A61B 5/00; A61B 5/0205; A61B 5/0215; A61M 25/09

(52) U.S. Cl. CPC A61B 5/6851; A61B 5/02055; A61M 2025/09175; A61M 25/09; A61M 2025/09083; A61B 5/02158

(57) ABSTRACT

An intravascular guidewire includes a flexible elongate member configured to be positioned within a vessel of a patient, the flexible elongate member comprising a proximal portion and a distal portion; an electronic component secured to the distal portion of the flexible elongate member; a first conductive member in communication with the electronic component; a second conductive member disposed at the proximal portion of the flexible elongate member; a tube disposed along a length of the flexible elongate member, the tube comprising a third conductive member, wherein the third conductive member is in electrical communication with the first and second conductive members.

### TECHNICAL FIELD

The present disclosure relates to intravascular devices, systems, and methods. In some embodiments, a guidewire includes a transition tube to facilitate electrical interconnection between a pressure sensor and electrical contacts at the proximal portion of the guidewire.

### BACKGROUND

Heart disease is very serious and often requires emergency operations. A main cause of heart disease is the accumulation of plaque inside the blood vessels, which eventually occludes the blood vessels. Common treatment options available to open up the occluded vessel include balloon angioplasty, rotational atherectomy, and intravascular stents. Traditionally, surgeons have relied on X-ray fluoroscopic images that are planar images showing the external shape of the silhouette of the lumen of blood vessels to guide treatment. Unfortunately, with X-ray fluoroscopic images, there is a great deal of uncertainty about the exact extent and orientation of the stenosis responsible for the occlusion, making it difficult to find the exact location of the stenosis. In addition, though it is known that restenosis can occur at the same place, it is difficult to check the condition inside the vessels after surgery with X-ray.

A currently accepted technique for assessing the severity of a stenosis in a blood vessel, including ischemia causing lesions, is fractional flow reserve (FFR). FFR is a calculation of the ratio of a distal pressure measurement (taken on the distal side of the stenosis) relative to a proximal pressure measurement (taken on the proximal side of the stenosis). FFR provides an index of stenosis severity that allows determination as to whether the blockage limits blood flow within the vessel to an extent that treatment is required. The normal value of FFR in a healthy vessel is 1.00, while values less than about 0.80 are generally deemed significant and require treatment.

Often intravascular guidewires are utilized to measure the pressure within the blood vessel. For a guidewire equipped with a pressure sensor, segments of electrical contacts are usually arranged at the proximal portion of the guidewire. Proper electrical connection between the pressure sensor and the electrical contacts is necessary for communication of signals between the pressure sensor and the electrical contacts.

### SUMMARY

Embodiments of the present disclosure provide an improved intravascular guidewire. The guidewire includes a pressure sensor at a distal portion. Electrical signals are transmitted between the pressure sensor and electrical contacts at the proximal portion of the guidewire via multiple metallic, conductive components. For example, electrical wires are connected to the pressure sensor. The proximal portion of the guidewire can include conductive members embedded within a polymer. A transition tube is positioned along a length of the guidewire. Conductive members are embedded within the transition tube. The electrical wires are directly connected to the conductive members of the transition tube. The conductive members at the proximal portion of the guide wire are directly connected to the conductive members of the transition tube. Accordingly, the pressure sensor is in electrical communication with the electrical contacts the proximal portion of the guidewire via the electrical wires, the conductive members of the transition tube, and the conductive members at the proximal portion of the guide wire. Utilizing the transition tube to establish electrical communication advantageously allows the electrical connections to be made while maintaining a small outer diameter of the guidewire, which makes it easier for the guidewire to more easily traverse tortuous vasculature of the patient.

Exemplary aspects of the present disclosure provide an intravascular guidewire, comprising: a flexible elongate member configured to be positioned within a vessel of a patient, the flexible elongate member comprising a proximal portion and a distal portion; an electronic component secured to the distal portion of the flexible elongate member; a first conductive member in communication with the electronic component; a second conductive member disposed at the proximal portion of the flexible elongate member; a tube disposed along a length of the flexible elongate member, the tube comprising a third conductive member, wherein the third conductive member is in electrical communication with the first and second conductive members.

In some aspects, the tube comprises a first polymer. In some aspects, the third conductive member is embedded within the first polymer. In some aspects, the flexible elongate member comprises a metal core, and wherein the tube is positioned around the metal core. In some aspects, the guidewire further includes a fourth conductive member disposed at the proximal portion of the flexible elongate member, wherein the fourth conductive member is directly in communication to the second conductive member. In some aspects, the first conductive member is directly in communication with the electronic component; the third conductive member is directly in communication with the first and second conductive members; and the electronic component is in communication with the fourth conductive member via the first, second, and third conductive members. In some aspects, the tube is disposed distally of the fourth conductive member. In some aspects, the tube is disposed distally of the third conductive member. In some aspects, the first conductive member comprises an electrical wire. In some aspects, the proximal portion of the flexible elongate member comprises a second polymer, and wherein the second conductive member is embedded within the second polymer. In some aspects, the guidewire further comprises: a plurality of first conductive members; a plurality of second conductive members; and a plurality of third conductive members. In some aspects, the flexible elongate member comprises a metal core, and a first of the plurality of conductive members is directly in communication with a first of the plurality of third conductive members; and a second of the plurality of conductive members is directly in communication with the metal core. In some aspects, a proximal portion of the tube comprises a first diameter less than a second diameter of a distal portion of the tube.

Exemplary aspects of the present disclosure provide a method of fabricating an intravascular guidewire, the method comprising: obtaining an electronic component and a first conductive member in communication with the electronic component; obtaining a flexible elongate member comprising a second conductive member; positioning a tube around at least a portion of the flexible elongate member, the tube comprising a third conductive member; and establishing communication between the electronic component and the second conductive member by electrically connecting the first conductive member to the third conductive member and electrically connecting the second conductive member to the third conductive member.

In some aspects, the flexible elongate member comprises a metal core, and wherein the positioning comprises positioning the tube around the metal core. In some aspects, the method further comprises exposing the third conductive member. In some aspects, the flexible elongate member further comprises a polymer surrounding the metal core, wherein the third conductive member is embedded within the polymer, and wherein the exposing comprises ablating the polymer. In some aspects, the method further includes reducing a diameter of a proximal portion of the tube. In some aspects, the exposing the third conductive member comprises exposing a proximal portion and a distal portion of the third conductive member, wherein the first conductive member is electrically connected to the distal portion of the third conductive member, and wherein the second conductive member is electrically connected to the proximal portion of the third conductive member. In some aspects, the electrically connecting comprises soldering.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic perspective view of an intravascular system, according to aspects of the present disclosure.

FIG. 2 is a diagrammatic side view of an intravascular device of the intravascular system of FIG. 1, according to aspects of the present disclosure.

FIG. 3 is a diagrammatic side view of a proximal connection portion of an intravascular device, according to aspects of the present disclosure.

FIG. 4 is a diagrammatic side view of a proximal connection portion and locking features of an intravascular device, according to aspects of the present disclosure.

FIG. 5 is a diagrammatic top view of an intravascular device, according to aspects of the present disclosure.

FIG. 6A is a diagrammatic perspective view of a proximal flexible elongate member, according to aspects of the present disclosure.

FIG. 6B is an enlarged diagrammatic perspective view of a portion of the proximal flexible elongate member indicated in FIG. 6A, according to aspects of the present disclosure.

FIG. 7 is a diagrammatic side view of a transition tube facilitating electrical interconnection, according to aspects of the present disclosure.

FIG. 8A is a diagrammatic side view of a transition tube facilitating electrical interconnection, according to aspects of the present disclosure.

FIG. 8B is a perspective view of a transition tube facilitating electrical interconnection, according to aspects of the present disclosure.

FIG. 8C is a diagrammatic side view of a transition tube facilitating electrical interconnection, according to aspects of the present disclosure.

FIG. 9 is a diagrammatic cross-sectional view along section lines 9-9 in FIGS. 7 and 8A, according to aspects of the present disclosure.

FIG. 10 is a diagrammatic cross-sectional side view of a connection region at the proximal portion the intravascular device, according to aspects of the present disclosure.

FIG. 11 is a flow diagram of a method of manufacturing the intravascular device, according to aspects of the present disclosure.

FIG. 12 is a perspective view of a proximal portion of the intravascular device during a stage of assembly, according to aspects of the present disclosure.

FIG. 13 is a perspective view of a proximal portion of the intravascular device during a stage of assembly, according to aspects of the present disclosure.

FIG. 14 is a perspective view of a proximal portion of the intravascular device during a stage of assembly, according to aspects of the present disclosure.

FIG. 15 is a perspective view of a proximal portion of the intravascular device during a stage of assembly, according to aspects of the present disclosure.

FIG. 16 is a perspective view of a proximal portion of the intravascular device during a stage of assembly, according to aspects of the present disclosure.

FIG. 17 is a perspective view of a proximal portion of the intravascular device during a stage of assembly, according to aspects of the present disclosure.

FIG. 18 is a perspective view of a proximal portion of the intravascular device during a stage of assembly, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that can be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, intravascular catheters and intravascular guidewires. In that regard, intravascular catheters may or may not include a lumen extending along its length for receiving and/or guiding other instruments. If the intravascular catheter includes a lumen, the lumen may be centered or offset with respect to the cross-sectional profile of the device.

In most embodiments, the flexible elongate members of the present disclosure include one or more electronic, optical, or electro-optical components. For example, without limitation, a flexible elongate member may include one or more of the following types of components: a pressure sensor, a flow sensor, a temperature sensor, an imaging element, an optical fiber, an ultrasound transducer, a reflector, a mirror, a prism, an ablation element, a radio frequency (RF) electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non-medical applications. However, some embodiments of the present disclosure are particularly suited for use in the context of human vasculature. Imaging of the intravascular space, particularly the interior walls of human vasculature can be accomplished by a number of different techniques, including ultrasound (often referred to as intravascular ultrasound (“IVUS”) and intracardiac echocardiography (“ICE”)) and optical coherence tomography (“OCT”). In other instances, infrared, thermal, or other imaging modalities are utilized. Further, in some instances the flexible elongate member includes multiple electronic, optical, and/or electro-optical components (e.g., pressure sensors, temperature sensors, imaging elements, optical fibers, ultrasound transducers, reflectors, mirrors, prisms, ablation elements, RF electrodes, conductors, etc.).

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As flexible elongate members can be solid, some embodiments of the present disclosure will include a housing portion at the distal portion for receiving the electronic components. Such housing portions can be tubular structures attached to the distal portion of the elongate member. Some flexible elongate members are tubular and have one or more lumens in which the electronic components can be positioned within the distal portion.

The electronic, optical, and/or electro-optical components and the associated communication lines are sized and shaped to allow for the diameter of the flexible elongate member to be very small. For example, the outside diameter of the elongate member, such as a guidewire or catheter, containing one or more electronic, optical, and/or electro-optical components as described herein are between about 0.0007″ (0.0178 mm) and about 0.118″ (3.0 mm), with some particular embodiments having outer diameters of approximately 0.014″ (0.3556 mm) and approximately 0.018″ (0.4572 mm)). As such, the flexible elongate members incorporating the electronic, optical, and/or electro-optical component(s) of the present application are suitable for use in a wide variety of lumens within a human patient besides those that are part or immediately surround the heart, including veins and arteries of the extremities, renal arteries, blood vessels in and around the brain, and other lumens.

“Connected” and variations thereof as used herein includes direct connections, such as being glued or otherwise fastened directly to, on, within, etc. another element, as well as indirect connections where one or more elements are disposed between the connected elements.

“Secured” and variations thereof as used herein includes methods by which an element is directly secured to another element, such as being glued or otherwise fastened directly to, on, within, etc. another element, as well as indirect techniques of securing two elements together where one or more elements are disposed between the secured elements.

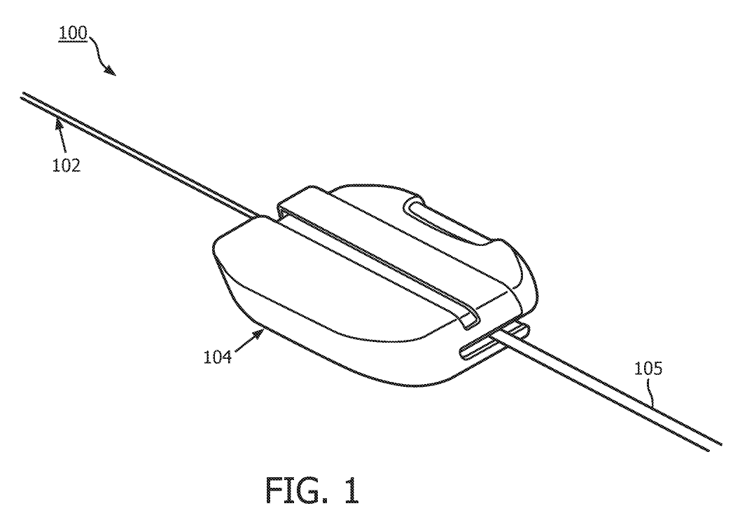

Referring initially to FIG. 1, shown therein is an intravascular system 100 according to an embodiment of the present disclosure. In that regard, the intravascular system includes an intravascular device 102 and a connector 104. As will be discussed in greater detail below, a communication cable 105 extends from the connector 104 in a direction coaxial with or parallel to the longitudinal axis of the intravascular device 102. As a result of the communication cable 105 extending coaxial with or parallel to the intravascular device, the connector 104 and communication cable 105 are less likely to catch on a patient, patient's clothing, medical equipment (including tubes, catheters, wires, leads, etc.) and/or other structures in the procedure room when maneuvering the intravascular device 102. In some instances, the connector 104 of the present disclosure incorporates one or more features of the connectors described in P.C.T. Application No. PCT/M2016/054528, titled “SIDE-LOADING CONNECTORS WITH INLINE CABLING FOR USE WITH INTRAVASCULAR DEVICES AND ASSOCIATED SYSTEMS AND METHODS” and filed Jul. 28, 2016, U.S. patent application Ser. No. 13/930,787, titled “SIDE-LOADING CONNECTORS FOR USE WITH INTRAVASCULAR DEVICES AND ASSOCIATED SYSTEMS AND METHODS” and filed Jun. 28, 2013, and U.S. patent application Ser. No. 13/930,636, titled “SIDE-LOADING CONNECTORS FOR USE WITH INTRAVASCULAR DEVICES AND ASSOCIATED SYSTEMS AND METHODS” and filed Jun. 28, 2013, each of which is hereby incorporated by reference in its entirety.

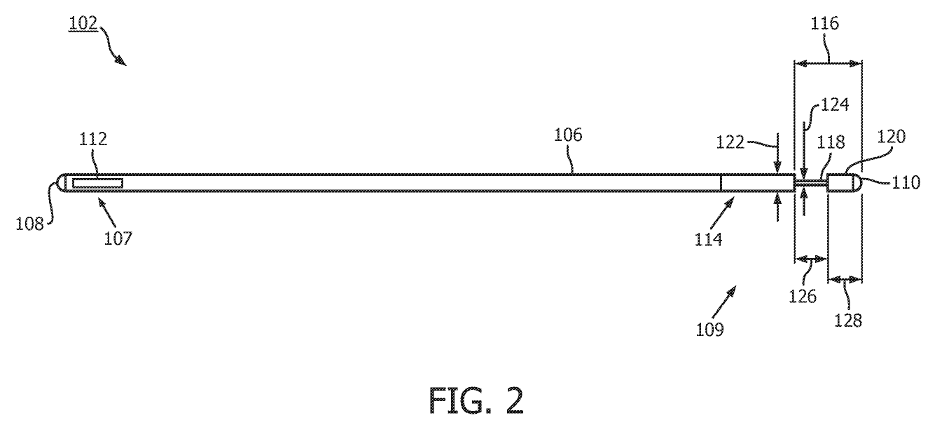

Referring now to FIG. 2, a side view of the intravascular device 102 is provided according to an embodiment of the present disclosure. As shown, the intravascular device 102 includes a flexible elongate member 106 having a distal portion 107 adjacent a distal end 108 and a proximal portion 109 adjacent a proximal end 110. A component 112 is positioned within the distal portion 107 of the flexible elongate member 106 proximal of the distal tip 108. Generally, the component 112 is representative of one or more electronic, optical, or electro-optical components. In that regard, the component 112 can include a pressure sensor, a flow sensor, a temperature sensor, an imaging element, an optical fiber, an ultrasound transducer, a reflector, a mirror, a prism, an ablation element, an RF electrode, a conductor, and/or combinations thereof. The specific type of component or combination of components can be selected based on an intended use of the intravascular device. In some instances, the component 112 is positioned less than 10 cm, less than 5, or less than 3 cm from the distal tip 108. In some instances, the component 112 is positioned within a housing of the intravascular device 102. In that regard, the housing can be a separate component secured to the flexible elongate member 106 in some instances. In other instances, the housing can be integrally formed as a part of the flexible elongate member 106.

The intravascular device 102 also includes a connection portion 114 adjacent the proximal portion 109 of the device. In that regard, the connection portion 114 can be spaced from the proximal end 110 of the flexible elongate member 106 by a distance 116. Generally, the distance 116 is between 0% and 50% of the total length of the flexible elongate member 106. While the total length of the flexible elongate member can be any length, in some embodiments the total length is between about 1300 mm and about 4000 mm, with some specific embodiments having a length of 1400 mm, 1900 mm, and 3000 mm. In some instances the connection portion 114 is spaced from the proximal end 110 between about 0 mm and about 1400 mm. In some specific embodiments, the connection portion 114 is spaced from the proximal end by a distance of 0 mm, 300 mm, and 1400 mm. Accordingly, in some instances the connection portion 114 is positioned at the proximal end 110. In some such embodiments, one or more aspects of the engagement and alignment features of the intravascular device 102 discussed below are positioned distal of the of the connection portion 114 instead of proximal of the connection portion 114 as shown in the embodiment of FIG. 2, or the engagement and alignment features may be omitted entirely.

In the illustrated embodiment of FIG. 2 the intravascular device 102 includes a locking section 118 extending proximally from the connection portion 114 to another section 120 that extends to proximal end 110. In the illustrated embodiment, the section 120 is rounded to proximal end 110. In other embodiments, the section 120 has a tapered, arcuate, and/or other changing profile as it extends proximally to proximal end 110. In that regard, in some instances the outer profile and/or diameter of the section 120 reduces as it extends proximally to proximal end 110 such that the reduced profile and/or diameter of the proximal end facilitates easier introduction of one or more other instruments over the intravascular device. In other embodiments, the section 120 has a constant profile as it extends proximally to proximal end 110. As section 120 is proximal to the locking section 118, it is sometimes referred to as the proximal section.

As shown, the connection portion 114 has a diameter 122 (or other similar measurement for outer cross-section profiles for non-circular cross-sectional embodiments) while locking section 118 has a diameter 124 (again, or other similar measurement for outer cross-section profiles for non-circular cross-sectional embodiments). The diameter 124 of locking section 118 is different than the diameter 122 of connection portion 114. In that regard, the different sizes of the diameters 122, 124 create a structure that is configured to facilitate alignment and/or connection of the intravascular device 102 to a connector, such as connector 104. In the illustrated embodiment, the diameter 124 of locking section 118 is less than the diameter 122 of the connection portion 114. In some embodiments, the diameter 124 of locking section 118 is between about 40% and about 80% of diameter 122, with some particular embodiments being about 42%, 64%, and/or other percentage of diameter 122. In that regard, in some embodiments the diameter 122 of connection portion 114 is between about 0.0178 mm and about 3.0 mm, with some particular embodiments being 0.3556 mm (0.014″) and 0.4572 mm (0.018″). Accordingly, in some embodiments the diameter 124 of locking section 118 is between about 0.007 mm and about 2.4 mm, with some particular embodiments being 0.15 mm, 0.19 mm, 0.23 mm, and 0.29 mm. In the illustrated embodiment, section 120 has a diameter that is approximately equal to diameter 122 and, therefore, greater than diameter 124. However, in other embodiments, section 120 has a diameter that is greater than diameter 122, less than diameter 122, greater than diameter 124, equal to diameter 124, and/or less than diameter 124. In some embodiments, locking section 118 is a section of a core wire extending through the connection portion 114. Locking section 118 and section 120 together can sometimes to be referred to as the locking feature.

As shown in FIG. 2, the locking section 118 extends proximally from connection portion 114 a distance 126, while section 120 extends proximally from locking section 118 to proximal end 110 a distance 128. Together, distances 126 and 128 equal the distance 116 that the connection portion 114 is spaced from the proximal end 110 of the intravascular device 102. In some instances, the distance 126 of is between about 0.508 mm (0.020″) and about 2.54 mm (0.10″), with some particular embodiments being 0.762 mm (0.030″), 1.016 mm (0.040″), and 1.524 mm (0.060″). Further, while the transition between connection portion 114 and locking section 118 and the transition between locking section 118 and section 120 are shown as being stepped in the illustrated embodiments, in other embodiments the transitions are tapered and/or otherwise make a gradual change in outer diameter along the length of the intravascular device. In some embodiments, use of tapered and/or gradual transitions results in the proximal portion of the intravascular device 102 not having any sharp edges. In some implementations, the use of tapered and/or gradual transitions for one or both of the transitions between locking section 118 and either the connection portion 114 or section 120 makes cleaning the proximal portion of the device (e.g., to remove any liquids or other unwanted materials on the surface of the proximal portion of the intravascular device) easier. In some embodiments, the intravascular system 100 can include one or more features described in U.S. patent application Ser. No. 15/374,312, titled “SIDE-LOADING CONNECTORS FOR USE WITH INTRAVASCULAR DEVICES AND ASSOCIATED SYSTEMS AND METHODS,” filed Dec. 9, 2016, which is hereby incorporated by reference in its entirety.

The connection portion 114 is configured to facilitate communication between the intravascular device 102 and another device. More specifically, in some embodiments the connection portion 114 is configured to facilitate communication of data obtained by the component 112 to another device, such as a computing device or processor. Accordingly, in some embodiments, the connection portion 114 includes one or more conductive portions. In some implementations, the connection portion 114 can include conductive bands, rings, coatings, coils, etc. In some instances, the connection portion 114 includes one or more electrical connectors, or conductive portions, as described in U.S. patent application Ser. No. 13/931,052, titled “INTRAVASCULAR DEVICES, SYSTEMS, AND METHODS,” filed Jun. 28, 2013, which is hereby incorporated by reference in its entirety. In other embodiments, the connection portion 114 includes an optical connector. In such instances, the connection portion 114 provides an optical connection to one or more optical communication pathways (e.g., fiber optic cable) that extend along the length of the flexible elongate member 106 and are optically coupled to the component 112. Further, in some embodiments the connection portion 114 provides both electrical and optical connections to both electrical conductor(s) and optical communication pathway(s) coupled to the component 112. In that regard, it should again be noted that component 112 can be comprised of a plurality of elements in some instances. In some instances, the connection portion 114 can be configured to provide a physical connection to another device, either directly or indirectly. In other instances, the connection portion 114 can be configured to facilitate wireless communication between the intravascular device 102 and another device. Generally, any current or future developed wireless protocol(s) may be utilized. In yet other instances, the connection portion 114 facilitates both physical and wireless connection to another device.

As noted above, in some instances the connection portion 114 provides a connection between the component 112 of the intravascular device 102 and an external device. Accordingly, in some embodiments one or more electrical conductors, one or more optical pathways, and/or combinations thereof extend along the length of the flexible elongate member 106 between the connection portion 114 and the component 112 to facilitate communication between the connection portion 114 and the component 112. Generally, any number of electrical conductors, optical pathways, and/or combinations thereof can extend along the length of the flexible elongate member 106 between the connection portion 114 and the component 112. In some instances, between one and ten electrical conductors (or conductive portions) and/or optical pathways extend along the length of the flexible elongate member 106 between the connection portion 114 and the component 112. For the sake of clarity and simplicity, the embodiments of the present disclosure described below include three electrical conductors and, therefore, the connection portion 114 is described as having three separate conductive portions corresponding to the three electrical conductors.

In some embodiments, the flexible elongate member 106 includes multiple core wires. For example, the flexible elongate member 106 can include a proximal core wire (or proximal metal core) and a distal core wire (or distal metal core) that are attached to one another. The components associated with the proximal portion of the intravascular device 102 (e.g., including the proximal core wire) can be referred to a proximal subassembly, and the components associated with the distal portion of the intravascular device 102 (e.g., including the distal core wire) can be referred to a distal subassembly. The flexible elongate member can refer to one or more components of the proximal subassembly and/or the distal subassembly. In some embodiments, the flexible elongate member 106 includes features as described in U.S. patent application Ser. No. 13/931,052, titled “INTRAVASCULAR DEVICE, SYSTEMS, AND METHODS” and filed Jun. 28, 2013, which is hereby incorporated by reference in its entirety.

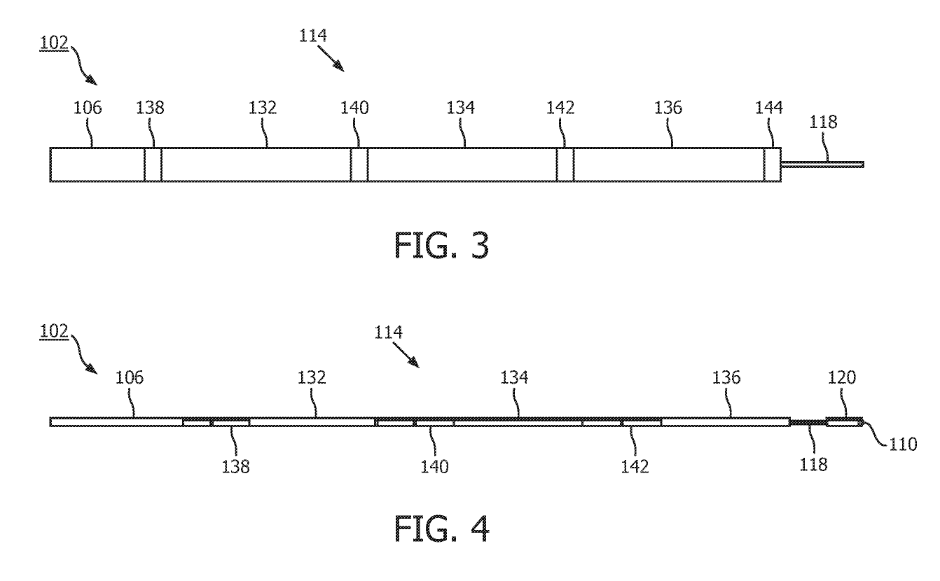

For example, as shown in FIG. 3, in some instances the connection portion 114 includes conductive portions 132, 134, and 136 that are separated from one another and the main body of the flexible elongate member 106 by insulating portions 138, 140, 142, and 144. In that regard, the conductive portions 132, 134, and 136 are formed of a conductive material and are portions of a hypotube, a coil, conductive ink, conductive coating formed over a tubular member, and/or combinations thereof in some instances. In some embodiments, the conductive portions 132, 134 and 136 include features as described in U.S. patent application Ser. No. 14/143,304, titled “INTRAVASCULAR DEVICES, SYSTEMS, AND METHODS” and filed Dec. 30, 2013, which is hereby incorporated by reference in its entirety. It is understood that the total number of communication pathways and/or the number of electrical conductors and/or optical pathways is different in other embodiments and, therefore, the number of conductive portions (or optical connectors) included in connection portion is different as well. More specifically, the number of communication pathways and the number of electrical conductors and optical pathways extending along the length of the flexible elongate member 106 can be selected based on the desired functionality of the component 112 and the corresponding elements that define component 112 to provide such functionality. As a result, the number and type of connections provided by connection portion 114 are likewise determined by the desired functionality of the component 112, the corresponding elements that define component 112 to provide such functionality, and the communication needs for such elements. Further still, in some instances, one or more of the insulating portions 138, 140, 142, and 144 is omitted. For example, as shown in the exemplary embodiment of FIG. 4, insulating portion 144 has been omitted.

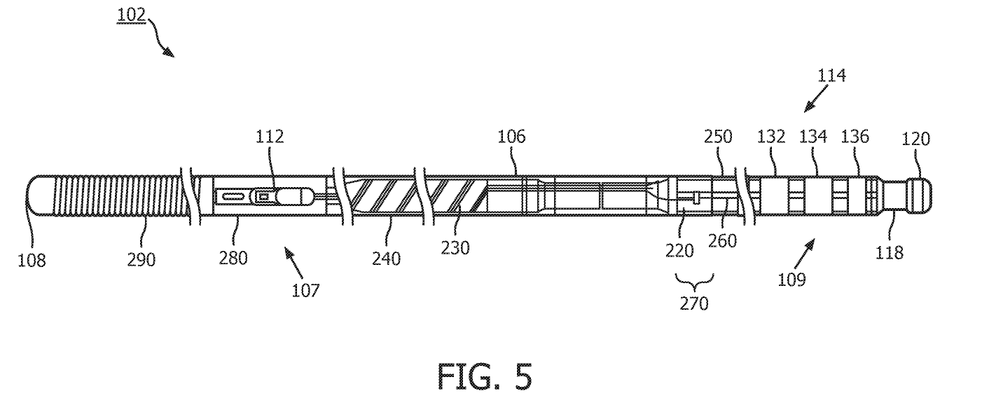

FIG. 5 is a top view of the intravascular device 102, according to aspects of the present disclosure. The intravascular device 102 can be an intravascular guidewire sized and shaped for positioning within a vessel of a patient. The intravascular device 102 can include the electronic component 112. For example, the electronic component 112 can be a pressure sensor configured to measure a pressure of blood flow within the vessel of the patient. The intravascular device 102 includes the flexible elongate member 106. The electronic component 112 is disposed at the distal portion 107 of the flexible elongate member 106. The electronic component 112 can be mounted at the distal portion 107 within a housing 280 in some embodiments. A flexible tip coil 290 extends between the housing 280 and the distal end 108. The connection portion 114 is disposed at the proximal portion 109 of the flexible elongate member 106. The connection portion includes the conductive portions 132, 134, 136. In some embodiments, the conductive portions 132, 134, 136 can be conductive ink that is printed and/or deposited around the flexible elongate member. In some embodiments, the conductive portions 132, 134, 136 are conductive, metallic rings that are positioned around the flexible elongate member. The locking section 118 and section 120 are disposed at the proximal portion 109 of the flexible elongate member 106.

The intravascular device 102 includes a distal core 210 and a proximal core 220. The distal core 210 and the proximal core 220 are metallic components forming part of the body of the intravascular device 102. For example, the distal core 210 and the proximal core 220 are flexible metallic rods that provide structure for the flexible elongate member 106. The diameter of the distal core 210 and the proximal core 220 can vary along its length.

In some embodiments, the intravascular device 102 comprises a distal assembly and a proximal assembly that are electrically and mechanically joined together, which results in electrical communication between the electronic component 112 and the conductive portions 132, 134, 136. For example, pressure data obtained by the pressure sensor 112 can be transmitted to the conductive portions 132, 134, 136. Control signals from a computer in communication with the intravascular device 102 can be transmitted to the electronic component 112 via the conductive portions 132, 134, 136. The distal subassembly can include the distal core 210. The distal subassembly can also include the electronic component 112, the conductive members 230, and/or one or more layers of polymer/plastic 240 surrounding the conductive members 230 and the core 210. For example, the polymer/plastic layer(s) can protect the conductive members 230. The proximal subassembly can include the proximal core 220. The proximal subassembly can also include one or more layers of polymer/plastic 250 surrounding the core 220 and/or conductive members 260 embedded within the one or more layers of polymer/plastic 250. In some embodiments, the proximal subassembly and the distal subassembly can be separately manufactured. During the assembly process for the intravascular device 102, the proximal subassembly and the distal subassembly can be electrically and mechanically joined together.

The intravascular device 102 includes one or more conductive members 230 in communication with the electronic component 112. For example, the conductive members 230 can be one or more electrical wires that are directly in communication with the electronic component 112. In some instances, the conductive members 230 are electrically and mechanically coupled to the electronic component 112 by, e.g., soldering. In some instances, the conductive members 230 comprise two or three electrical wires (e.g., a bifilar cable or a trifilar cable). An individual electrical wire can include a bare metallic conductor surrounded by one or more insulating layers. The conductive members 230 can extend along the length of the distal core 210. For example, at least a portion of the conductive members 230 can be spirally wrapped around the distal core 210.

The intravascular device 102 includes one or more conductive members 260 at the proximal portion of the flexible elongate member 106. The conductive members 260 are embedded within polymer/plastic layer(s) 250. The conductive members 260 are directly in communication with the conductive portions 132, 134, and/or 136. In some instances, the conductive members 230 are electrically and mechanically coupled to the electronic component 112 by, e.g., soldering. In some instances, the conductive portions 132, 134, and/or 136 comprise conductive ink (e.g., metallic nano-ink, such as silver or gold nano-ink) that is deposited or printed directed over the conductive members 260.

As described herein, electrical communication between the conductive members 230 and the conductive members 260 can be established at the connection region 270 of the flexible elongate member 106. By establishing electrical communication between the conductive members 230 and the conductive members 260, the conductive portions 132, 134, 136 can be in electrically communication with the electronic component 112.

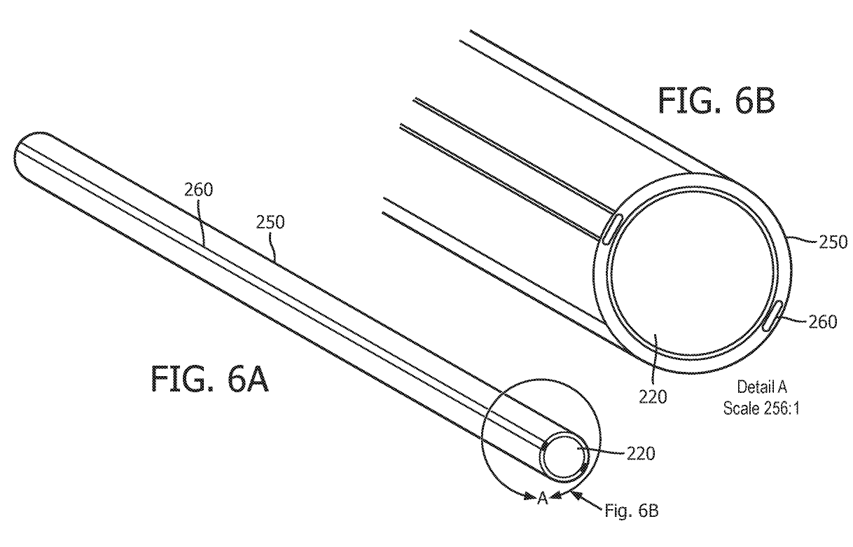

FIGS. 6A and 6B illustrate perspective views of a proximal subassembly of the intravascular device 102. The proximal subassembly includes including the proximal core wire 220. The core wire 220 can be made of a metal or metal alloy in some embodiments, such as nickel-cobalt base alloy (e.g., MP35N). The diameter of the core wire 220 can be between 0.0100 and 0.0110, including values such as 0.0105″, 0.0107″, 0.0109″ in some embodiments. The one or more polymer/plastic layers 250 surround the core wire 220. The layer 250 can include polyimide in some embodiments. One or more metallic, conductive members 260 are embedded within the polymer/plastic layer(s) 250. For example, the proximal subassembly can include two conductive members 260. The polymer/plastic layer(s) 250 can electrically isolate the conductive members 260 from the metallic core wire 220. Any suitable process (e.g. grinding, ablating, etc.) for removing a portion of the polymer/plastic layer(s) 250 can be used to expose portions of the conductive members 260. Electrical communication with the conductive members 260 can be established at the exposed portions of the conductive members 260. In some embodiments, the proximal subassembly can include features as described in U.S. patent application Ser. No. 14/143,304, titled “INTRAVASCULAR DEVICES, SYSTEMS, AND METHODS” and filed Dec. 30, 2013, which is hereby incorporated by reference in its entirety. In some aspects, due to the large diameter core wire, there is minimal space available to make the electrical connection between the composite wire ribbons 260 and the trifilar 230 while ensuring adequate sealing against fluid ingress and maintaining a small outer diameter of the intravascular device 102.

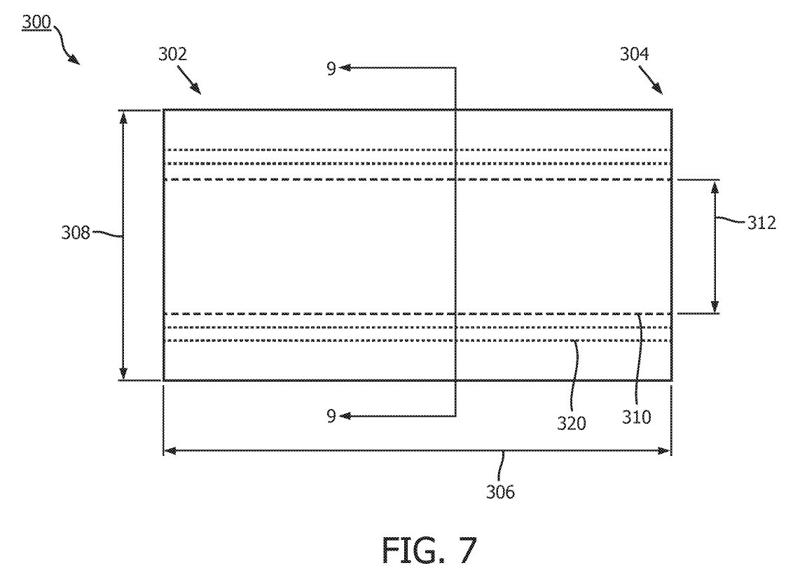

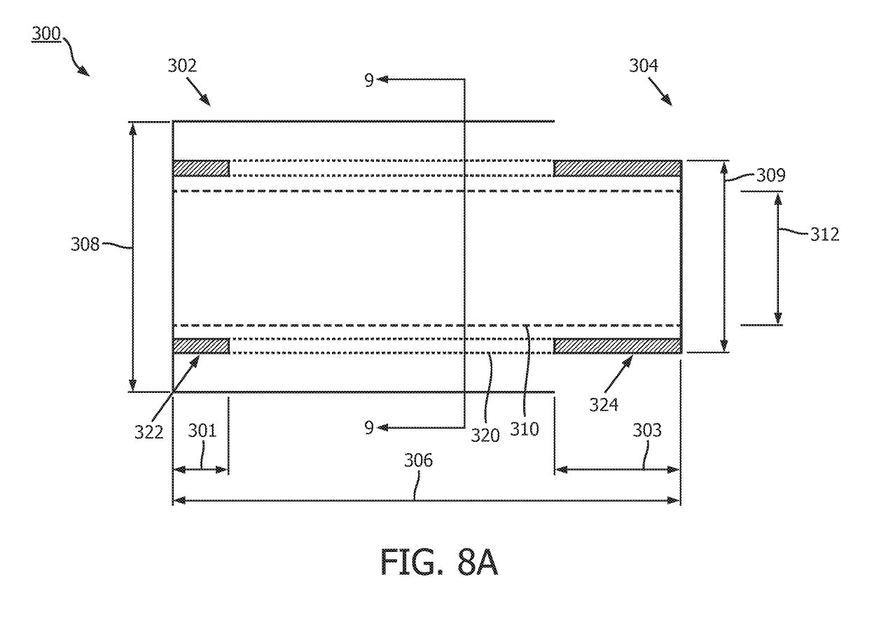

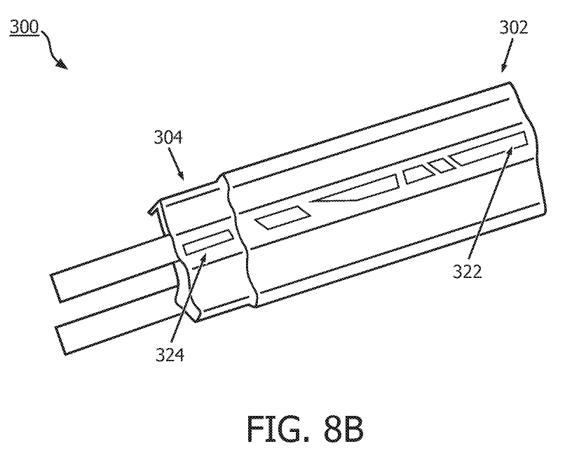

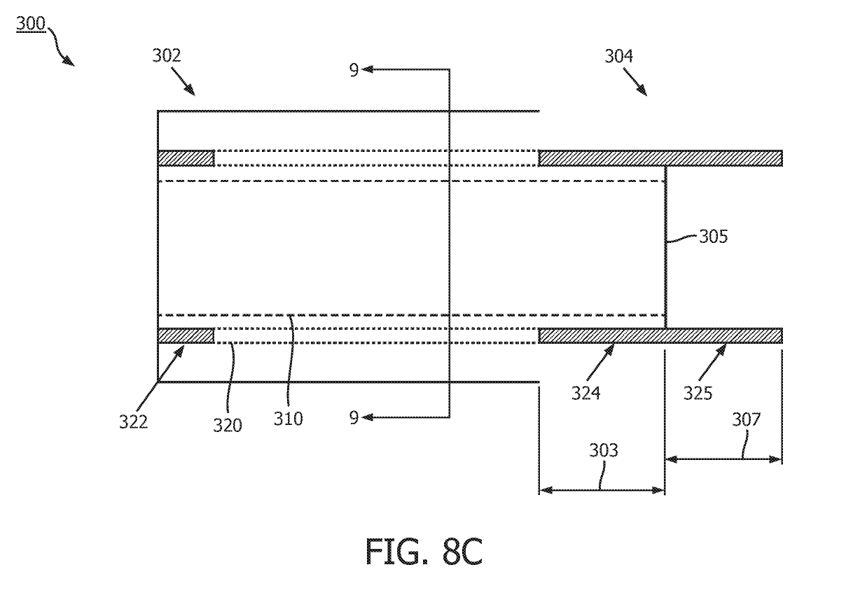

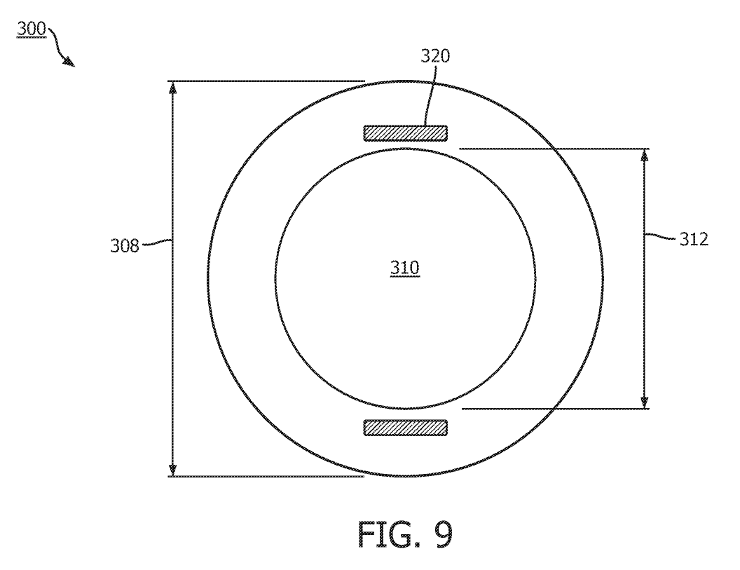

FIGS. 7, 8A, 8B, and 9 illustrate a tube 300 facilitating electrical communication between the conductive members 230 and the conductive members 260. The tube 300 can be positioned along a length of the flexible elongate member 106. In some instances, the tube 300 can be referenced as a transition composite tube. FIG. 7 is a diagrammatic side view of the tube 300. FIG. 8A is a diagrammatic side view of the tube 300 after portions of the tube 300 have been removed during the manufacturing process. FIG. 8B is a including a perspective view of the tube 300. FIG. 9 is a cross-sectional view along section lines 9-9 in FIGS. 7 and 8A.

The tube 300 can be generally cylindrical with an annular cross-sectional shape. The tube 300 can be formed of one or more layers of polymer and/or plastic. For example, the tube 300 can be formed of polyimide. The tube 300 includes a distal portion 302 and a proximal portion 304. Note that the orientation of FIG. 8B is reversed relative to FIGS. 7 and 8A. In FIGS. 7 and 8A, the distal portion 302 is on the left side of the drawing, while in FIG. 8B, the distal portion 302 is on the right side of the drawing. Similarly, in FIGS. 7 and 8A, the proximal portion 304 is on the right side of the drawing, while in FIG. 8B, the proximal portion 304 is on the left side of the drawing.

The tube 300 can fabricated in a continuous process and then cut into small component lengths. An individual length is illustrated in the FIG. 7. FIG. 7 illustrates the tube 300 at an earlier stage in the manufacturing process. As shown in FIGS. 7, 8A, and 9, the tube 300 includes one or more embedded conductive members 320. The tube 300 can include two conductive members, for example. The conductive members 320 are metallic components that are surrounded by the polymer/plastic of the tube 300. A lumen 310 extends through the entire length of the tube 300. In some embodiments, a diameter 312 of the lumen 310 can be between 0.005″ and 0.02″, including values such as 0.0086″, 0.011″, 0.018″and/or other suitable values. The diameter 312 of the lumen 310 can be referenced as an inner diameter of the tube 300. The diameter 312 can be large enough to accommodate the proximal core wire 220, e.g., reduced diameter portion of the proximal core wire 220 in some embodiments. In some embodiments, an outer diameter 308 of the tube can be between 0.005″ and 0.028″, including values such as 0.012″, 0.015″, and 0.025″, and/or other suitable values. In some embodiments, the conductive ribbons 320 are embedded within the polymer/plastic of the tube 300 with a bias toward the inner diameter 312 to ensure adequate space for electrical connection to the trifilar 230.

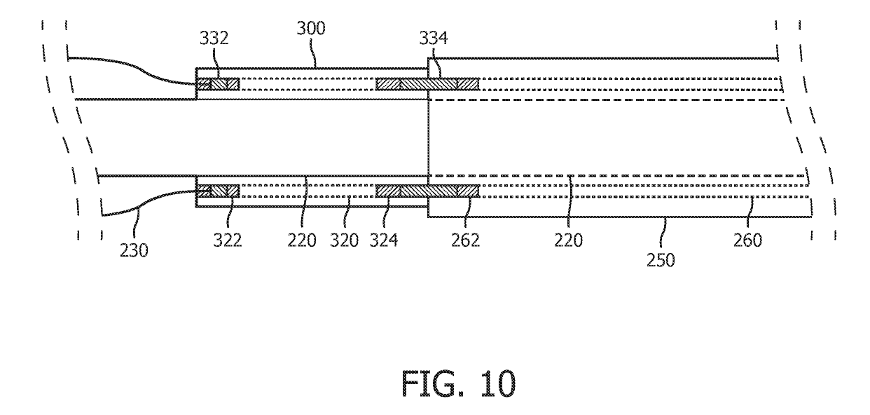

FIGS. 8A and 8B illustrate the tube 300 at a later stage in the manufacturing process. Relative to FIG. 7, for example, material has been removed the distal portion 302 and the proximal portion 304 of the tube 300. For example, the distal and proximal portions 302, 304 have been laser ablated during the manufacturing process. Any suitable machining and/or shaping process can be used to remove material from and/or shape the tube 300 and/or the proximal subassembly (e.g., the polymer/plastic layer 250 of FIG. 10), such as rolling, grinding, laser ablation, electrical discharge machining (EDM), and lathing. As a result, portions of the polymer/plastic have been removed, and portions of the conductive members 320 have been exposed. For example, a distal portion 322 and a proximal portion 324 of the conductive member 320 have been exposed. The exposed distal portion 322 can have length 301 between 0.004 and 0.015″, including values such as 0.010″, and/or other suitable values. The exposed proximal portion 324 can have length 303 between 0.002″ and 0.010″, including values such as 0.005″, and/or other suitable values.

Different amounts of material from the tube 300 can be removed at the distal portion 302 and the proximal portion 304. For example, ablation of the polyimide at the proximal portion 304 completely exposes a short section 324 of both ribbons 320 and also thins out or reduces the diameter of the proximal portion 304. The reduced diameter 309 of the proximal portion 304 can be between 0.007″ and 0.020″, includes values such as 0.010″, 0.013″, 0.016″, 0.020″, 0.023″, and/or other suitable values. The reduced diameter 309 of the proximal portion 304 allows expansion as the tube 300 is pushed onto the core wire 220. For example, the core wire 220 can transition from a smaller diameter to a larger diameter, and the tube 300 can expand as the tube 300 is pushed onto the transition. Narrow ablation slots 322 are also made along the distal portion 302 and are contained within a width of each ribbon 320. The slots 322 allow for soldering of the trifilar 230 to the exposed ribbon 320 in the slot 322. The portions of the conductive members 320 that are indicated by dashed marks in FIG. 8A are unexposed. For example, the polymer/plastic of the tube 300 covers the portions of the conductive members 320 that are indicated by dashed marks in FIG. 8A.

FIG. 8C illustrate an exemplary embodiment of the tube 300 in which a section 325 of the conductive member 320 extends proximally beyond the step-down polymide area at the proximal portion 304 of the tube 300 and a proximal end of the tube 305. The step-down section of the tube 300 exposes the proximal portion 324 of the ribbon 320, but the polymer/plastic of tube 300 still exists below the ribbon 320. The isolated ribbons 325 have nothing beneath them because all polyimide of the tube 300 has been removed. Advantageously, the isolated ribbons 325 may allow for the conductive member 320 to be positioned closer to and/or in contact with the conductive member 260 of the proximal subassembly (e.g., FIG. 10) to allow for better electrical and/or mechanical interconnection between the conductive members 320 and 260 without an increase in diameter resulting from the body of the tube 300. A length 307 of the isolated ribbons 325 can be between 0.004″ to 0.015″, including values such as 0.010″ and/or other suitable values. In some embodiments, the distal portion 322 of the conductive member 320 can end distally beyond a distal end 311 of the tube 300. In some embodiments, isolated portions of the conductive member 320 (e.g., unsupported by any polymer/plastic of the tube 300) can be disposed at the distal portion 304, the proximal portion 302, and/or both the proximal and distal portions 302, 304 of the tube 300.

FIG. 10 illustrates a cross-sectional side view of the connection region 270 of the intravascular device 102. The tube 300 is positioned around the core wire 200 and adjacent to the proximal subassembly, including the core wire 220, the polymer/plastic layer(s) 250, and the embedded conductive members 260. For example, a portion of the core wire 200 extends through the lumen 310 of the tube 300. The tube 300 facilitates electrical connection between the conductive members 230 and the conductive members 260. As a result, the electronic component 112 is in electrical communication with the conductive portions 132, 134, 136. The conductive member 230 is directly in communication with the conductive member 320. For example, the bare conductor of the electrical wire 230 can be electrically and mechanically coupled to the exposed portion 322 of the conductive member 320 by solder 332. The conductive member 230 is directly in communication with the conductive member 260. For example, the exposed portion 324 of the conductive member 320 can be electrically and mechanically coupled to the exposed portion 262 of the conductive member 260 by solder 334. Each conductive member 230 can be in communication with a respective conductive member 320. Similarly, each conductive member 320 can be in communication a respective conductive member 260. In embodiments in which the electronic component 112 is in communication with the trifilar 230, two of electrical wires of the trifilar 230 can be in communication with respective conductive portions 320, and the third electrical wire of the trifilar 230 can be in communication with the core wire 220. For example, the core wire 220 can serve as electrical ground for the electronic component as a result of electrical communication between the third electrical wire of the trifilar 230 and the core wire 220. In some embodiments, the outer diameter of the tube 300 can be smaller than the outer diameter of the proximal subassembly (e.g., the polymer/plastic layer 250).

In some embodiments, the tube 300 is disposed distally of the fourth conductive member (e.g., the conductive portions 132, 134, 136). In some embodiments, the tube is disposed distally of the third conductive member (e.g., the conductive members 260).

Utilizing the tube 300 to establish electrical communication between the conductive members 230 and the conductive members 260 advantageously allows the electrical connections to be made in the minimal space that is available, as a result of the large core wire 220, while ensuring sealing against fluid ingress and maintaining a small outer diameter of the intravascular device 102. For example, the transition composite tube 300 provides a space-saving electrical connection to the proximal composite core ribbons 260 while also providing increased space for the electrical connection to the trifilar 230 from the distal core 210. The more difficult, space-sensitive connection to the composite core ribbons 260 can be accomplished and verified as a subassembly on the composite core 220 prior to attaching the distal subassembly. The polyimide of the tube 300 acts as an insulating material that also withstands the high heat required for soldering connections.

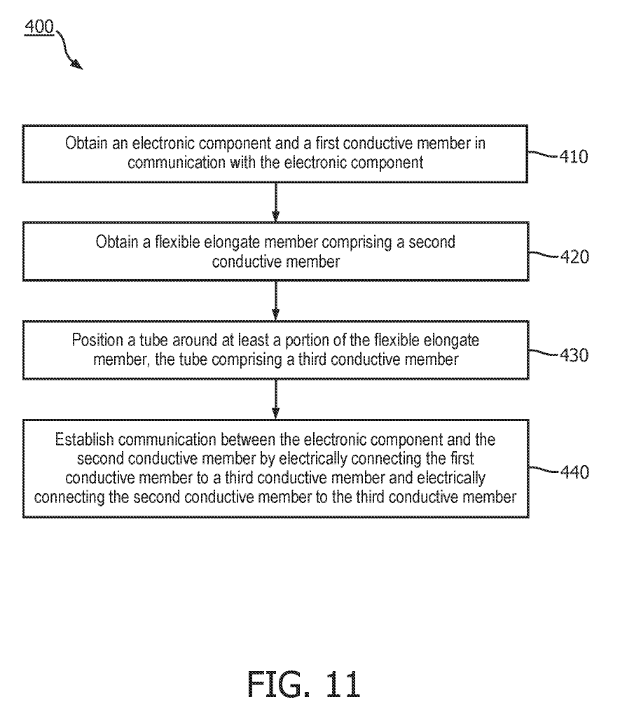

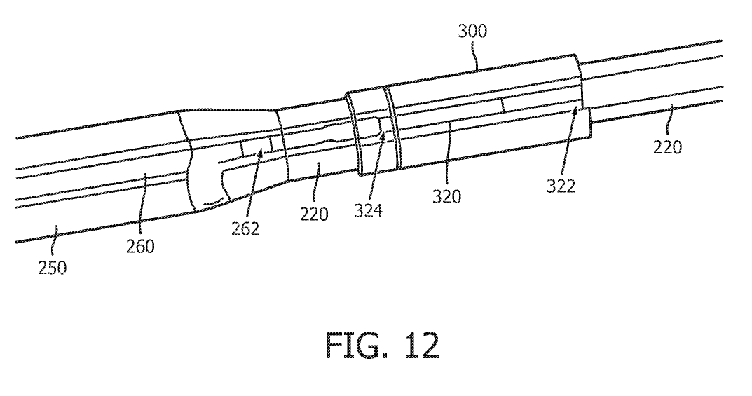

Referring now to FIG. 11, shown therein is a flowchart of method 400 for fabricating intravascular device 102. It is understood that the steps of method 400 may be performed in a different order than shown in FIG. 11, additional steps can be provided before, during, and after the steps, and/or some of the steps described can be replaced or eliminated in other embodiments. The steps of the method 400 can be carried out by a manufacturer of the intravascular device 102. One or more steps of the method 400 can be described with reference to one or more of FIGS. 12-18.

At step 410, the method 400 includes obtaining an electronic component and a first conductive member in communication with the electronic component. The electronic component can be the electronic component 112, such as a pressure sensor, in some embodiments. The first conductive member can be an electrical wire of the trifilar cable 230, for example. In some embodiments, the method 400 includes obtaining all or portions of the distal subassembly. For example, the method 400 can include obtaining the electronic component mounted to the distal core wire 210. The method 400 can include assembling one or more components of the distal subassembly, such as securing the electronic component 112 to the distal core wire 210, wrapping the conductive members 230 around the distal core wire, etc.

At step 420, the method 400 includes obtaining a flexible elongate member comprising a second conductive member. For example, the flexible elongate member can be a proximal subassembly, including the proximal core wire 220, the polymer/plastic layer(s) 250, and the one or more embedded conductive members 230. The second conductive member can be the conductive member 230. The method 400 can include assembling one or more components of the proximal subassembly.

At step 430, the method 400 includes positioning a tube around at least a portion of the flexible elongate member, the tube comprising a third conductive member. The tube can be the tube 300 including the one or more embedded conductive member 320. The third conductive member can be the conductive member 320. FIG. 12 is a perspective view of a proximal portion of the intravascular device 102 during assembly. In particular, FIG. 12 illustrates the tube 300 being slid proximally towards the exposed portion 262 of the conductive member 260. The tube 300 is positioned around the core wire 220. For example, the core wire 220 extends through the lumen of the tube 300. The proximal subassembly includes the conductive members 260 embedded within the plastic/polymer layer 250. The method 400 can include removing a portion of the layer 250 to expose a portion 262 of the conductive member 260. The method 400 can include exposing the third conductive member. For example, the method 400 can include removing a portion of the tube 300 to expose the proximal portion 324 and the distal portion 322 of the conductive member 320. The exposing can include ablating the polymer layer. In some embodiments, the method 400 includes reducing a diameter of a proximal portion of the tube, e.g., by ablating the polymer/plastic layer of the tube 300.

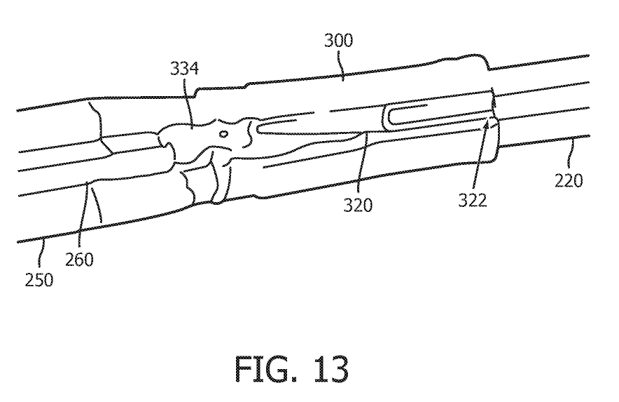

At step 440, the method 400 includes establishing communication between the electronic component and the second conductive member by electrically connecting the first conductive member to a third conductive member and electrically connecting the second conductive member to the third conductive member. For example, the method 400 includes electrically connecting the first conductive member to the exposed distal portion of the third conductive member. The method 400 includes electrically connecting the second conductive member to the proximal portion of the third conductive member. FIG. 13 is a perspective view of a proximal portion of the intravascular device 102 during assembly. As shown in FIG. 13, the method 400 can include applying solder across the exposed portion 262 of the conductive member 260 and the exposed portion 324 of the conductive member 320. This electrically and/or mechanically connects the conductive member 260 and the conductive member 320.

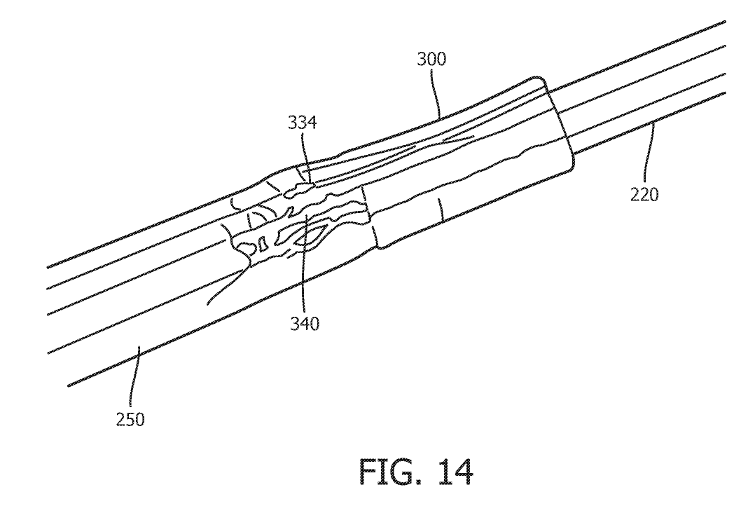

FIG. 14 is a perspective view of a proximal portion of the intravascular device 102 during assembly. As shown in FIG. 14, the method 400 can include applying adhesive 340, such as an epoxy, across the junction between the proximal subassembly (e.g., the polymer/plastic layer 350, the proximal core 220) and the tube 300, including the solder 334. The adhesive 340 further mechanically couples the proximal subassembly and the tube 300.

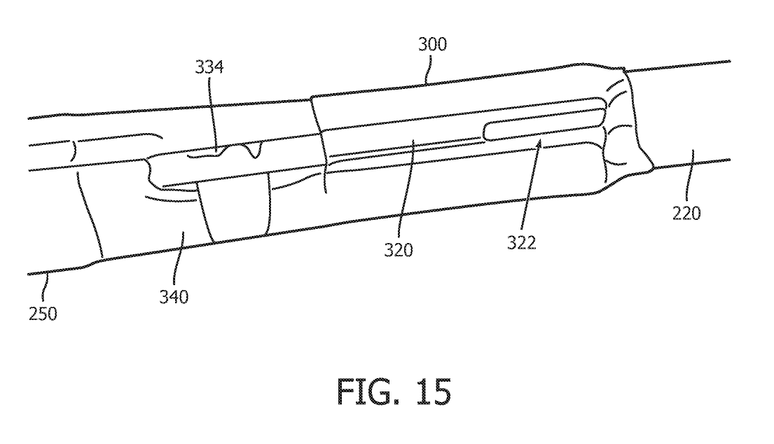

FIG. 15 is a perspective view of a proximal portion of the intravascular device 102 during assembly. After the adhesive applied (as illustrated in FIG. 14), the method 400 can include allowing the adhesive to wick in. The method 400 can also include curing the adhesive, such as by application of heat or light. The tube 300 includes the exposed distal portion 322 of the conductive member 320.

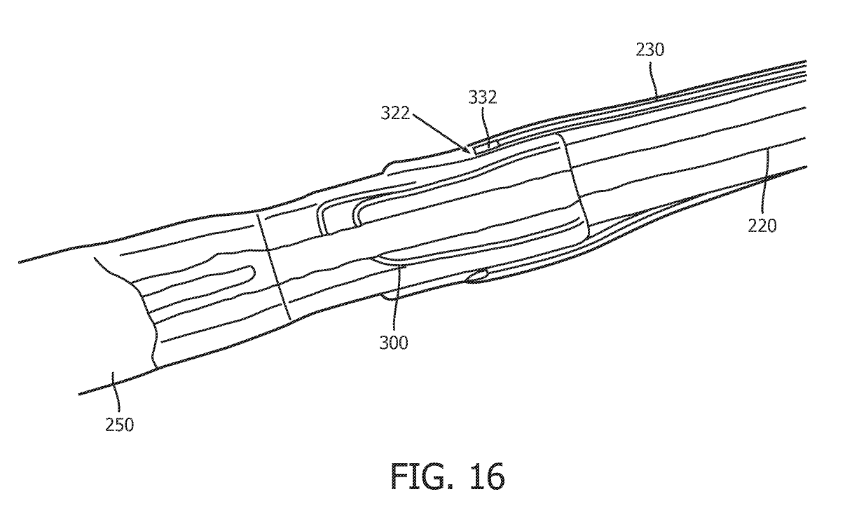

FIG. 16 is a perspective view of a proximal portion of the intravascular device 102 during assembly. As shown in FIG. 16, the method 400 can include applying solder 332 while the conductive member 230 is positioned on the exposed portion 322 of the conductive member 320. This electrically and/or mechanically connects the conductive member 230 and the conductive member 320. A conductive member 230 can be coupled to the corresponding conductive member 320.

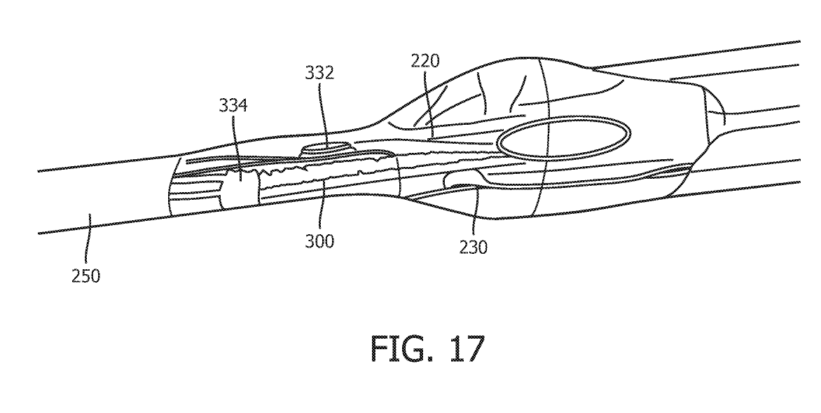

FIG. 17 is a perspective view of a proximal portion of the intravascular device 102 during assembly. As shown in FIG. 17, the method 400 can include positioning a shrink tube (e.g., a fluorinated ethylene propylene or FEP shrink tube) around the proximal portion of the intravascular device 102, such as around a portion of the core wire 220 and/or the conductive member 230. The method 400 can include filling the shrink tube with an adhesive, such as epoxy. The method 400 can include shrinking the tube, such as by applying heat. The method 400 can include curing the adhesive, such as by applying heat or light. The method 400 can include removing the shrink tube in some embodiments.

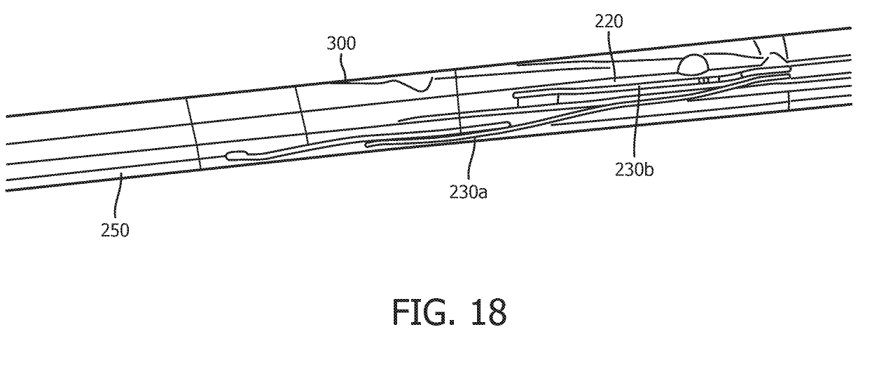

FIG. 18 is a perspective view of a proximal portion of the intravascular device 102 during assembly. The method 400 can include positioning a thin-walled (between 0.00015″ and 0.0002″) shrink tube (e.g., a polyethylene terephthalate or PET shrink tube) around the proximal portion of the intravascular device 102. The method 400 can include filling the shrink tube with an adhesive, such as epoxy. The shrink tube can be positioned such that it overlaps the electrical interconnection between the conductive member 230 and the conductive member 320 and/or the electrical interconnection between the conductive member 320 and the conductive member 260. The method 400 can include shrinking the tube, such as by applying heat. The method 400 can include curing the adhesive, such as by applying heat or light. The conductive member 230a is connected to the corresponding conductive member of the tube 300. The conductive member 230b is connected to core wire 220.

The method 400 can include forming the fourth conductive members at the proximal portion of the flexible elongate member. For example, the fourth conductive members can be directly in communication with the conductive members 260. For example, the conductive members 260 can be mechanically and/or electrically coupled to the fourth conductive members. In some embodiments, the fourth conductive members can be rings that are slid over the flexible elongate members. In some embodiments, the fourth conductive members are conductive ink that is deposited or printed around the flexible elongate member. The method 400 can include joining the proximal and distal subassemblies in some embodiments.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intravascular guidewire, comprising:
a flexible elongate member configured to be positioned within a vessel of a patient, the flexible elongate member comprising a proximal portion and a distal portion;
an electronic component secured to the distal portion of the flexible elongate member;
a first conductive member in communication with the electronic component;
a second conductive member disposed at the proximal portion of the flexible elongate member;
a tube disposed along a length of the flexible elongate member, the tube comprising a third conductive member, wherein the third conductive member is in electrical communication with the first and second conductive members.

2. The intravascular guidewire of claim 1,
wherein the tube comprises a first polymer.

3. The intravascular guidewire of claim 2,
wherein the third conductive member is embedded within the first polymer.

4. The intravascular guidewire of claim 1, wherein the flexible elongate member comprises a metal core, and wherein the tube is positioned around the metal core.

5. The intravascular guidewire of claim 1, further comprising a fourth conductive member disposed at the proximal portion of the flexible elongate member, wherein the fourth conductive member is directly in communication to the second conductive member.

6. The intravascular guidewire of claim 5, wherein:
the first conductive member is directly in communication with the electronic component;
the third conductive member is directly in communication with the first and second conductive members; and
the electronic component is in communication with the fourth conductive member via the first, second, and third conductive members.

7. The intravascular guidewire of claim 5, wherein the tube is disposed distally of the fourth conductive member.

8. The intravascular guidewire of claim 1, wherein the tube is disposed distally of the third conductive member.

9. The intravascular guidewire of claim 1, wherein the first conductive member comprises an electrical wire.

10. The intravascular guidewire of claim 1, wherein the proximal portion of the flexible elongate member comprises a second polymer, and wherein the second conductive member is embedded within the second polymer.

11. The intravascular guidewire of claim 1, further comprising:
a plurality of first conductive members;
a plurality of second conductive members; and
a plurality of third conductive members.

12. The intravascular guidewire of claim 11, wherein the flexible elongate member comprises a metal core, and wherein:
a first of the plurality of conductive members is directly in communication with a first of the plurality of third conductive members; and
a second of the plurality of conductive members is directly in communication with the metal core.

13. The intravascular guidewire of claim 1, wherein a proximal portion of the tube comprises a first diameter less than a second diameter of a distal portion of the tube.

14. A method of fabricating an intravascular guidewire, the method comprising:
obtaining an electronic component and a first conductive member in communication with the electronic component;
obtaining a flexible elongate member comprising a second conductive member;
positioning a tube around at least a portion of the flexible elongate member, the tube comprising a third conductive member; and
establishing communication between the electronic component and the second conductive member by electrically connecting the first conductive member to the third conductive member and electrically connecting the second conductive member to the third conductive member.

15. The method of claim 14, wherein the flexible elongate member comprises a metal core, and wherein the positioning comprises positioning the tube around the metal core.

16. The method of claim 15, further comprising:
exposing the third conductive member.

17. The method of claim 16, wherein the flexible elongate member further comprises a polymer surrounding the metal core, wherein the third conductive member is embedded within the polymer, and wherein the exposing comprises ablating the polymer.

18. The method of claim 17, further comprising:
reducing a diameter of a proximal portion of the tube.

19. The method of claim 17, wherein the exposing the third conductive member comprises exposing a proximal portion and a distal portion of the third conductive member, wherein the first conductive member is electrically connected to the distal portion of the third conductive member, and wherein the second conductive member is electrically connected to the proximal portion of the third conductive member.

20. The method of claim 14, wherein the electrically connecting comprises soldering.
